(19) United States
(12) Patent Application Publication
(10) Pub. No.: US 2024/0203590 A1
(43) Pub. Date: Jun. 20, 2024

(54) SYSTEM AND METHOD FOR DETECTION OF HEALTH-RELATED BEHAVIORS

(71) Applicant: THE TRUSTEES OF DARTMOUTH COLLEGE, Hanover, NH (US)

(72) Inventors: DAVID KOTZ, Lyme, NH (US); SHENGJIE BI, WHITE RIVER JUNCTION, VT (US)

(21) Appl. No.: 18/287,428

(22) PCT Filed: May 2, 2022
(86) PCT No.: PCT/US22/27344

§ 371 Date: Oct. 18, 2023

Related U.S. Application Data
(60) Provisional application No. 63/182,938, filed on May 2, 2021.

(51) Int. Cl. G16H 50/20; G06V 10/764; G06V 10/82

(52) U.S. Cl. CPC G16H 50/20; G06V 10/764; G06V 10/82

(57) ABSTRACT

A method of detecting health-related behaviors, comprising training a model with video of the mouths of one or more users, capturing video using a camera focused on a user's mouth; processing the video using the model; and outputting one or more health-related behaviors detected in the captured video by the model. A method of training the model includes preprocessing a video captured by a camera focused on a user's mouth by extracting raw video frames and optical flow features; classifying the video frame-by-frame; aggregating video frames in sections based on their classifications; and training the model using the classified and aggregated video frames. A wearable device for capturing video of a user's mouth is also described.

### CROSS-REFERENCE TO RELATED APPLICATIONS

This application claims priority to Provisional Patent Application No. 63/182,938 filed May 2, 2021, titled “System and Method for Detection of Health-Related Behaviors,” incorporated herein by reference.

### GOVERNMENT RIGHTS

This invention was made with government support under grant nos. CNS-1565269 and NSF CNS-1835983 awarded by the National Science Foundation. The government has certain rights in the invention.

### BACKGROUND

Chronic disease is one of the most pressing health challenges faced in the United States, and around the world. According to one report, nearly half (approximately 45%, or 133 million) of all Americans suffer from at least one chronic disease, and the number is growing. Chronic diseases are a tremendous burden to the individuals, their families, and to society. By 2023, diabetes alone is estimated to cost $430 billion to the US economy. The onset or progression of diseases like obesity, hypertension, diabetes, lung cancer, heart disease and metabolic disorders are strongly related to eating behavior. Scientists are still trying to fully understand the complex mixture of diet, exercise, genetics, sociocultural context, and physical environment that can lead to these diseases. One of these factors, diet is one of the most challenging to measure, i.e., recognizing eating behaviors in free-living conditions that is accurate, automatic, and seamless.

The detection of health-related behaviors (such as eating, drinking, smoking, coughing, sniffling, laughing, breathing, speaking, and face touching) is the basis of many mobile-sensing applications for healthcare and can help trigger other kinds of sensing or inquiries. Wearable sensors may be used for mobile sensing due to their low cost, ease of deployment and use, and ability to provide continuous monitoring. Among wearable sensors, head-mounted devices are ideal for detecting these health-related behaviors because they are physically close to where these behaviors happen, particularly in a real-world, free-living environment as opposed to a more artificial, lab-based environment.

Fit and comfort is important for gathering accurate data from a user in a free-living environment. A device that is uncomfortable will not be worn for a length of time needed to gather valid data, or may be adjusted for comfort in such a way that the camera is not focused on the area of interest. The design of the device is also important for capturing behaviors that vary somewhat, but are considered the same for the purpose of classification, such as meal and snack scenarios, for example.

### SUMMARY OF THE EMBODIMENTS

In a first aspect, a method of training a model to detect health-related behaviors, includes preprocessing a video captured by a camera focused on a user's mouth by extracting raw video frames and optical flow features: classifying the video frame-by-frame; aggregating video frames in sections based on their classifications; and training the model using the classified and aggregated video frames.

In a second aspect, a method of detecting health-related behaviors, comprising training a model using the method of the first aspect, capturing video using a camera focused on a user's mouth; processing the video using the model; and outputting health-related behaviors detected in the captured video by the model.

In a third aspect, a wearable device for inferring health-related behaviors in real-life situations, includes a housing adapted to be worn on a user's head: a camera attached to the housing, the camera positioned to capture a video of a mouth of the user: a processor for processing the video: a memory for storing the video and instructions for processing the video: wherein the processor executes instructions stored in the memory to: preprocess a video captured by a camera focused on a user's mouth: classify the video frame-by-frame using a target frame and a plurality of frames preceding the target frame: aggregate video frames in sections based on their classifications; and output an inferred health-related behavior of each segment of the captured video.

### BRIEF DESCRIPTION OF THE FIGURES

FIG. 1 depicts a head-mounted device worn by a user, in an embodiment.

FIG. 2A depicts representative video frames recorded by the device of claim 1 during eating periods, in an embodiment.

FIG. 2B depicts representative video frames recorded by the device of claim 1 during non-eating periods, in an embodiment.

FIG. 3 is a flowchart illustrating a method for processing video captured by a camera focused on a user's mouth, in embodiments.

### DETAILED DESCRIPTION OF THE EMBODIMENTS

In embodiments, a head-mounted camera uses a computer-vision based approach to detect health-related behaviors such as eating behaviors. Throughout this disclosure, health-related behaviors and eating behaviors may be used interchangeably. More generally, a wearable system and associated method is used to automatically detect when people eat, and for how long, in real-world, free-living conditions. Although embodiments are shown in terms of a head-mounted device for detecting eating, methods and systems discussed herein may also be used with cameras mounted on other devices, such as a chest-mounted device or a necklace. Any mounting device may be used so long as the camera has a view of the user's mouth and doesn't impede activities such as eating, drinking, smoking, coughing, sniffling, laughing, breathing, speaking, and face touching. Further, a head-mounted device may take the form of a baseball cap, visor, or any hat with a brim, for example, so long as the hat includes a portion that extends some distance away from the wearer's face and allows for the mounting of a camera. Any reference herein to a cap should be understood as encompassing any of the head-mounted devices discussed above.

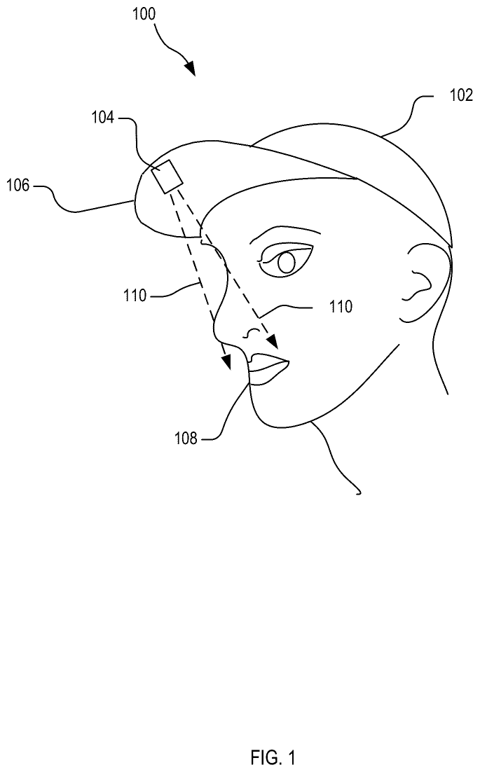

FIG. 1 shows a user 100 wearing a head-mounted device in the form of cap 102. A camera 104 is fixed under brim 106 of cap 102. Camera 104 is positioned to capture video of mouth 108 of user 100 as shown by dotted lines 110. Camera 104 records video during the user's normal daily activities. Video may be recorded continuously or sporadically in response to a trigger. In embodiments, video is recorded but not audio. This reduces a data processing burden and enhances the privacy of a user wearing the device.

In embodiments, cap 102 also includes other elements such as control circuitry and a battery that are not shown. These elements may be positioned in various locations on cap 102 to enhance comfort and ease of use. For example, control circuitry may be positioned on the upper part of brim 106 and connected to a battery positioned on the back of cap 102. Control circuitry and one or more batteries may be placed in the same location inside or outside cap 102. Devices may be attached to cap 102 in a temporary or more permanent manner. Further, video data captured by camera 104 may be sent wirelessly to another user device, such as a smartwatch or cell phone.

Camera 104 may be used to collect data about the eating behavior of user 100. In embodiments, cap 102 and camera 104 may be used in diverse environments. In embodiments, camera 104 records a video having a resolution approximately 360p (640×360 pixels) and a frame rate of approximately 30 frames per second (FPS), although other resolutions and frame rates may be used depending on processing capability and other factors.

Video captured by camera 104 may be processed using computer-vision analysis to provide an accurate way to of detecting eating behaviors. Convolutional Neural Networks (CNNs) may be used for image recognition and action recognition in videos. A method of processing video captured by a head-mounted camera to infer health-related behaviors using a CNN includes training the CNN model using test data, then using the trained model to infer behaviors.

In embodiments, training data acquisition includes having participants eat various types of food including, for example, rice, bread, noodles, meat, vegetables, fruit, eggs, nuts, chips, soup, and ice cream while wearing cap 102 or another device for capturing video. Participants recorded data in diverse environments including houses, cars, parking lots, restaurants, kitchens, woods, and streets.

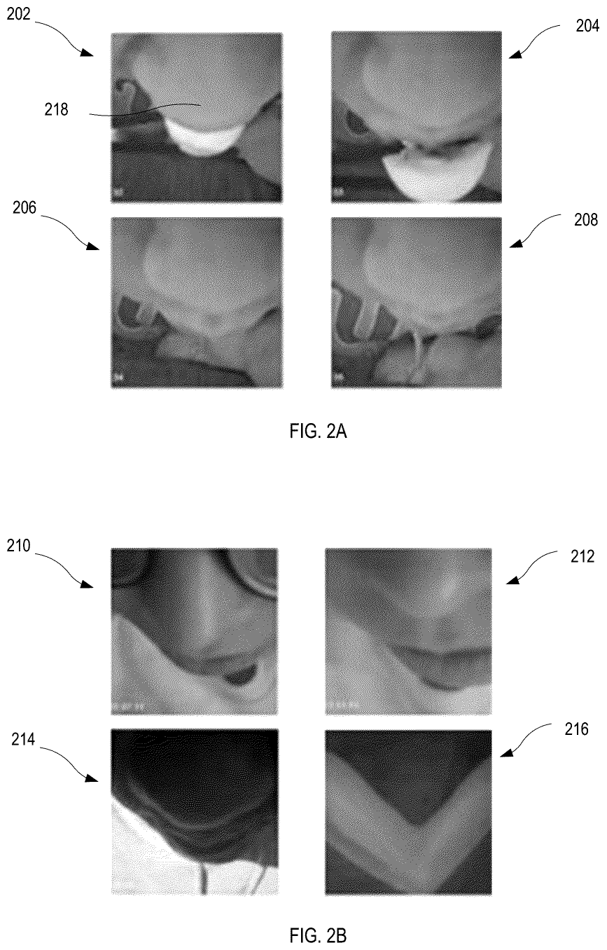

FIGS. 2A and 2B show examples of video frames recorded during eating and non-eating periods, respectively. FIG. 2A shows views 202, 204, 206 and 208 of a user in the act of eating various foods. Facial features, such as nose 218 are also visible in all four views. FIG. 2B shows views 210, 212, 214 and 216 of a user that were recorded during non-eating periods. Facial features, such as nose 218 and glasses 220 are visible. While the lighting conditions in all four views of FIG. 2A are similar, there is more variability between the lighting conditions in view: 210, 212, 214 and 216.

Captured videos are annotated so accuracy of inferences may be evaluated. In embodiments, an annotation process may include multiple steps such as execution, audit and quality inspection. In the execution step, an annotator watched the video and annotated each period of eating, at a 1-second resolution. Thus, for every second in the video, the annotator indicated whether the individual was eating or not. Next, the audit step, an auditor watched the video and checked whether the annotations were consistent with the content in the video. The auditor noted any identified inconsistency for the next step: quality inspection. Finally, in the third step, a quality inspector reviewed the questionable labels and made the final decision about each identified inconsistency. The quality inspector also conducted a second-round inspection of 20% of the samples that were considered consistent during the previous two inspection rounds. Although a representative example of video annotation is disclosed, other processes are contemplated.

Training and using a model for inferring a user's eating behavior includes a number of processes. Functions are described as distinct processes herein for purposes of illustration. Any process may be combined or separated into additional processes as needed. We next describe our evaluation metrics, and the stages of our data-processing pipeline: preprocessing, classification, and aggregation.

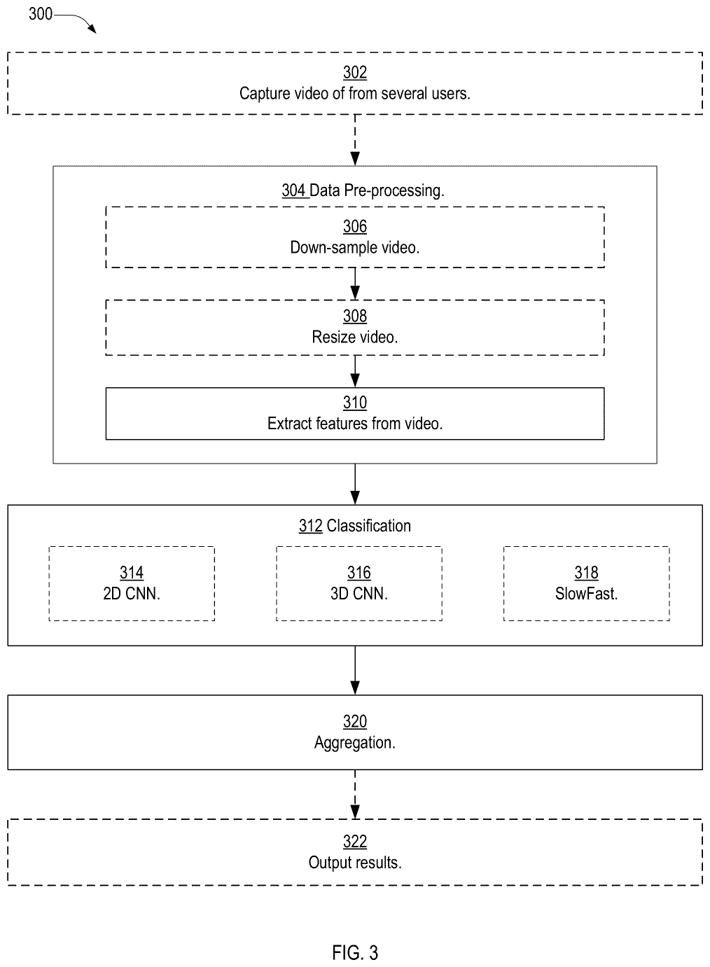

FIG. 3 is a flowchart a method 300 for using video captured by a camera focused on a user's mouth to train a Convolution Neural Network (CNN) model, in embodiments. Once trained, the CNN model may be used to analyze and detect health-related behaviors such as eating. Method 300 includes steps 304, 310, 312 and 320, wherein step 312 includes one of steps 314, 316 or 318. In embodiments, method 300 also includes at least one of steps 302, 306, 308 and 322.

Step 302 includes capturing video of a user's mouth. In an example of step 302, a camera 104 mounted on brim 106 of a cap 102 is used to capture video of a user's mouth 108. In embodiments, a video comprises a series of frames having a representative resolution and frame rate of approximately 360p (640×360 pixels) and 30 frames per second (FPS), although other resolutions and frame rates are contemplated. When training the CNN model, a video dataset is collected from several users for a period of time long enough to encompass both eating and non-eating periods, for example, 5 hours. The video dataset captured in step 302 may be divided into three subsets: training, validation, and test. The training subset for training the CNN models discussed herein, the validation subset for tuning the parameters of these models, and the test subset for evaluation. The ratio of the total duration of videos is approximately 70:15:15, although any ratio may be used to satisfy the goals of training, tuning and evaluation a CNN model.

### Data Preprocessing

In step 304, captured video data is pre-processed to reduce subsequent computational burden. Step 304 may include one or more of substeps 306, 308 and 310. For example, steps 306 and 308 may not be needed. In step 306, the captured video is down-sampled to reduce the number of frames per second of video. In an example of step 306, video is down-sampled from 30 FPS to 5 FPS. Other frame rates are contemplated.

In step 308, the captured video is resized. In an example of step 308, the video is resized from camera dimensions of 640×360 pixels to 256×144 pixels. Because CNN models usually take inputs in square shape, and to further reduce the memory burden, the down-sampled videos were cropped to extract the central 144×144 pixels. Steps 306 and 308 may not be needed depending on the camera used, the resources available to the developer for training, the devices used in the cap and other factors. Further, a camera with a sensor that captures square images natively may be used, or a CNN variant may be developed that works with rectangular images.

In step 310, features are extracted from the down-sampled and resized video. In an example of step 310, for the cropped videos, raw video frames (appearance feature) and optical flow (motion feature) are extracted and stored in a record format optimized for faster model training speed, for example, a tensorflow record. In embodiments, three RGB channels were used for raw video frames. A Dual TV-L1 optical flow may be used because it can be efficiently implemented on a modern graphics processing unit (GPU). The optical flow is calculated based on the target frame and the frame directly preceding it, and produces two channels corresponding to the horizontal and vertical components.

### Classification

Step 312 includes classifying each video frame as eating or non-eating. In an example of step 312, different CNN architectures may be used. Although three representative CNN architectures are shown, other CNN models are contemplated. As used herein, a SlowFast CNN refers to any type of two-stream video analysis that recognizes that motion and semantic information in a video change at different rates and can be processed in different streams, or pathways. In general, small CNN models with relatively few parameters may be used to enable deployment of the models on wearable platforms.

Step 314 represents a 2D CNN, step 316 represents a 3D CNN and step 318 represents a Slow Fast CNN. Method In embodiments, the CNN models output a probability of eating for each frame (every 0.2 seconds). Table 1 illustrates parameters chosen for model specification. The 2D CNN and 3D CNN models use a the five-layer CNN architecture, which includes 4 conventional layers (each with a pooling layer after) and 1 fully connected (dense) layer. For the SlowFast model, there is 1 more fusion layer between the last pooling layer and the fully connected layer to combine the slow and fast pathways (see Table 1).

TABLE 1
CNN model specification. In the columns under the 2D CNN heading, bold and italic
show the difference between using frame and flow. In the columns under the SlowFast
heading, bold and italic show the difference between the slow and fast pathways.

| | 2D CNN (frame or flow) | | | 3D CNN | | | SlowFast (slow + fast) | | |
|---|---|---|---|---|---|---|---|---|---|
| | | Kernel | | | Kernel | | | Kernel | |
| Layer | Dimension | size | Stride | Dimension | size | stride | dimension | size | stride |
| data | 128² × 3\|2 | | | 16 × 128² × 3 | | | 4 × 128²× 3 | | |
| | | | | | | | 16 × 128²× 3 | | |
| conv1 | 128² × 32 | 3² | 1² | 16 × 128² × 32 | 3 × 3² | 1 × 1² | 4 × 128²× 32 | 1\|3 × 3² | 1 × 1² |
| | | | | | | | 16 × 128²× 8 | | |
| pool 1 | 64² × 32 | 2² | 2² | 8 × 64² × 32 | 2 × 2² | 2 x 2² | 4 × 64²× 32 | 1 × 2² | 1 × 2² |
| | | | | | | | 16 × 64²× 8 | | |
| conv2 | 64² × 32 | 3² | 1² | 8 × 64² × 32 | 3 × 3² | 1 × 1² | 4 × 64²× 32 | 1\|3 × 3² | 1 × 1² |
| | | | | | | | 16 × 64²× 8 | | |
| pool2 | 32² x 32 | 2² | 2² | 4 × 32² × 32 | 2 × 2² | 2 × 2² | 4 × 32²× 32 | 1 × 2² | 1 × 2² |
| | | | | | | | 16 × 32²× 8 | | |
| conv3 | 32² x 32 | 3² | 1² | 4 × 32² × 32 | 3 × 3² | 1 × 1² | 4 × 32²× 64 | 1\|3 × 3² | 1 × 1² |
| | | | | | | | 16 × 32²× 16 | | |
| pool3 | 16² × 32 | 2² | 2² | 1 × 16² × 32 | 2 × 2² | 2 × 2² | 4 × 16²× 64 | 1 × 2² | 1 × 2² |
| | | | | | | | 16 × 16²× 16 | | |
| conv4 | 16² × 32 | 3² | 1² | 2 × 16² × 32 | 3 × 3² | 1 × 1² | 4 × 16²× 64 | 1\|3 × 3² | 1 × 1² |
| | | | | | | | 16 × 16²× 16 | | |
| pool4 | 8² × 3 2 | 2² | 2² | 1 × 8² × 32 | 2 × 2² | 2 × 2² | 4 × 8²× 64 | 1 × 2² | 1 × 2² |
| | | | | | | | 16 × 8²× 16 | | |
| fusion | | | | | | | 8² × 64 | | |
| flatten | 4096 | | | 4096 | | | 4096 | | |
| dense | 1024 | | | 1024 | | | 1024 | | |
| dense | 2 | | | 2 | | | 2 | | |

### 2D CNN

Step 314 includes processing video data using a 2D CNN. In an example of step 314, two types of input features may be used: raw video frames or precalculated optical flows. When using raw video frames as input features, the CNN model makes predictions based on the appearance information extracted from only one image segmented from videos (i.e., one video frame); the CNN model produces one inference for each frame, independently of its classification of other frames. Since the 2D CNN model is simpler than the other two models—it uses only one frame or optical flow as the input—it will use less memory and computation power when deploying on wearable. Additionally, the 2D CNN functions as a baseline, indicating what is possible with only appearance information or motion information. Max pooling is used for all the pooling layers.

### 3D CNN

Step 316 includes processing video data using a 3D CNN. In an example of step 316, a 3D CNN has the ability to learn spatio-temporal features as it extends the 2D CNN introduced in the previous section by using 3D instead of 2D convolutions. The third dimension corresponds to the temporal context. For purposes of illustration, in a representative example, the input of 3D CNN consists of the target frame and the 15 frames preceding it (3 seconds at 5 FPS), which is a sequence of 16 frames in total. In other words, the 3D CNN considers a consecutive stack of 16 video frames. Other parameters are contemplated. The output of the CNN model is the prediction for the last frame of the sequence (the target frame). To take maximum advantage of the available training data, we generated input using a window shifting by one frame. In embodiments, temporal convolution kernels of size 3 and max pooling for temporal dimension in all the pooling layers are used.

### SlowFast

Step 318 includes processing video data using a Slow Fast model. Similarly to the 3D CNN, the Slow Fast model also considers a temporal context of the previous frames preceding the target frame, but the Slow Fast model processes the temporal context at two different temporal resolutions. In embodiments, Slow Fast parameters were chosen to be the factors a=4, temporal kernel size 3 for the fast pathway, and 9=0.25, temporal kernel size 1 for the slow pathway.

### Model Training Policy

In embodiments, any of the models disclosed above may use an Adam optimizer to train each model on the training set. A batch size of 64 based on the memory size of the cluster may be used but other sizes are contemplated. In embodiments, training may run for 40 epochs with a learning rate starting at 2×10⁻⁴ and exponentially decaying at a rate of 0.9 per epoch, for example.

In embodiments, cross entropy for loss calculation may be used for all models. Due to the nature of the eating data collected from users in a real-world environment, the classes tend to be imbalanced with more non-eating instances than eating instances. During a model training phase, this imbalance may be corrected by scaling the weight of loss for each class using the reciprocal of number of instances in each class. In a representative example, in a batch of training samples (size 64) with 54 non-eating instances and 10 eating instances, the ratio of weight of loss between non-eating class and eating class may be 10.

To avoid over fitting, method 300 discussed herein uses L2 loss with a lambda of 1×10⁻⁴ for regularization and applied dropout in all models on convolutional and dense layers with rate 0.5. Additionally, early stopping may be included if the model yields are observed with increasing validation errors at the end of the training stage. In embodiments, data augmentation is used by applying random transformations to the input: cropping to size 128×128, horizontal flipping, small rotations, brightness, and contrast changes. All models may be learned end to end.

We note cropping is performed to reduce data volume to enhance processing speed and is useful for our particular hardware and software environment. In other embodiments having faster processors or larger power budgets, cropping may not be necessary and higher resolutions maintained. In embodiments having lower resolution cameras, cropping may also be avoided.

### Aggregation

Step 320 includes aggregating the prediction results of classification step 312. In an example of step 320, an aggregation rule is applied to sections of video: if more than 10% of the frames in a minute were labeled eating, that minute of video is labeled as eating. Since the CNN models output predictions every 0.2 seconds (one prediction per frame), after aggregation, the resolution of eating-detection results may be 1 minute in both cases.

Table 2 summarizes the resulting performance metrics for eating detection with a 1-minute resolution using the four models. The best result is achieved using SlowFast model, with an F1 score of 78.7% and accuracy of 90.9%.

TABLE 2
Performance metrics for eating detection with CNN models.

| | #Param- | Accu- | | | F1 |
|---|---|---|---|---|---|
| Model | eters | racy | Precision | Recall | Score |
| 2D CNN (with frame) | 4.26M | 71.0% | 38.3% | 49.8% | 43.3% |
| 2D CNN (with flow) | 4.26M | 78.3% | 46.9% | 67.8% | 55.4% |
| 3D CNN | 4.39M | 86.4% | 72.4% | 75.3% | 73.8% |
| SlowFast | 4.49M | 90.9% | 75.5% | 82.2% | 78.7% |

To assess the usefulness of temporal context, the accuracy of various models discussed herein may be compared with and without temporal context. As shown in Table 2, the 3D CNN model (F1 score 73.8%) outperforms 2D CNN with frame (F1 score 43.3%) and 2D CNN with flow (F1 score 55.4%). The SlowFast model also outperforms 2D CNN (with frame) and 2D CNN (with flow) by more than 23% F1 score. As shown by Table 2, temporal context for eating detection in the field considerably improves model performance. Using only spatial information (either frame (appearance) or flow (motion) feature) from one single video frame may be not sufficient for achieving good eating-detection performance.

Table 2 also appears to show that precision is the worst score across all the metrics for all the four models. The low precision may indicate that there were many false positives (the model indicated eating and ground truth indicated non-eating). Some of the reasons for false positives may be include behaviors such as talking, drinking, blowing one's nose, putting on face masks, mouth rinsing, wiping one's mouth with a napkin, unconscious mouth or tongue movement, and continuously touching one's face or mouth. Additional training data and deeper CNN networks may be used to reduce false positives.

### Head-Mounted Device

Design of a device for use with the method disclosed herein involves balancing computational, memory and power constraints with comfort and wearability of a mobile or wearable platform in real-world environments. As discussed herein and shown in Table 2, both the 3D CNN and SlowFast models achieved better performance than the 2D CNN models for eating detection. However, the SlowFast model is a fusion of two 3D CNN models so it may require more computational resources than a single 3D CNN model. The various dimensions given below with regard to processing speed, memory size and power consumption, for example, are for purposes of illustrating principles as discussed herein, other dimensions are contemplated.

The computational resources needed for a deep-learning model are often measured in gigaflops, i.e., 1×10⁹ floating point operations per second (GFLOPS). In embodiments, a 3D CNN model having 8 convolutional layers may be estimated to require from 10.8 to 15.2 GFLOPS, after compression with different pruning algorithms. As disclosed herein, a 3D CNN model with 4 convolutional layers would likely require less than 10.8 GFLOPS after pruning. GPUs are used in modern mobile or wearable platforms such as smartphones, smart watches and similar wearable platforms. A platform selected for device as disclosed herein should include enough computing resources to run a 3D CNN model for inference.

The memory needed for running the 3D CNN models include at least two parts: storing the raw video frame sequence, and storing the model parameters. The pixel values of RGB images are integers and the model parameters are floating-point numbers, which, in embodiments, are both 4 bytes each. Using the data dimensions from Table 1, the memory needed for storing the raw video frame sequence is 16×1282×3×4=3.15 MB. Using the parameters from Table 2, the memory needed for storing the parameters of 3D CNN models is 4.39×4=17.56 MB. Hence the memory needed for running the 3D CNN models is at about 3.15+17.56=20.71 MB, and should fit easily in a mobile platform with 32 MB of main memory.

The power consumption of the system includes of at least two parts: the camera (to capture images or videos) and the processor (to run the CNN model). In embodiments, an ultra-low power CMOS camera with parameters as disclosed herein (96×96 pixels, 20 FPS) consumes less than 20 μW, for example. GPU devices may also be selected to operate below a maximum power threshold. The performance of a system and method for detection of health-related behaviors may be adjusted to minimize power consumption by detecting certain circumstances of a user, such as sitting at a desk for a period of time. During periods of minimal movement, the system may be set to a sleep or idle mode.

Changes may be made in the above methods and systems without departing from the scope hereof. Although embodiments are disclosed for detecting eating, with enough training data and proper model tuning, the method and system disclosed herein has potential to generalize from eating detection to the detection of other health-related behaviors (such as drinking, smoking, coughing, sniffling, laughing, breathing, speaking, and face touching). As many of these behaviors are short and infrequent during normal daily life, inference may need large-scale field studies and substantial video annotation effort to collect enough training data.

Methods and systems disclosed herein use RGB videos frames with a relatively low resolution (144×144 pixels) and low frame rate (5 FPS) due to limited computation resources. Different key parameters (i.e., frame rate, frame resolution, color depth) that affect cost (e.g., power consumption) and performance (e.g., F1 score) may also be used.

A fusion of visual and privacy-sensitive audio signals may be incorporated into any of the methods and systems disclosed herein and may yield better performance in eating detection. Acoustic-based Automatic Dietary Monitoring (ADM) systems for eating detection use audio signals (e.g., chewing sound and swallowing sound) to detect eating behaviors. As head-mounted cap 102 is located close to a user's face, camera 104 may be modified to capture both video and audio signals. An on-board module that processes audio on the fly may address this issue.

As disclosed herein, a system and method for detecting health-related behaviors uses a traditional digital camera and Computer Vision (CV) techniques. Other types of cameras (e.g., thermal cameras and event cameras) may also be useful sensors for eating detection. Thermal cameras could take advantage of the temperature information from food and use it as a cue for eating detection. Event cameras contain pixels that independently respond to changes in brightness as they occur. Compared with traditional cameras, event cameras have several benefits including extremely low latency, asynchronous data acquisition, high dynamic range, and very low power consumption, which make them interesting sensors to explore for eating and health-related behavior detection.

Further, a deeper CNN or a CNN with different parameters than those described above may improve the performance of eating detection, including reducing the occurrence of false positives.

### Combinations of Features

Features described above as well as those claimed below may be combined in various ways without departing from the scope hereof. The following enumerated examples illustrate some possible, non-limiting combinations:

(A1) A method of training a model to detect health-related behaviors, includes preprocessing a video captured by a camera focused on a user's mouth by extracting raw video frames and optical flow features: classifying the video frame-by-frame: aggregating video frames in sections based on their classifications; and training the model using the classified and aggregated video frames.

(A2) In method (A1), preprocessing further comprises, before extracting raw video frames and optical flow features down-sampling the video to reduce a number of frames per second; and resizing the video to a square of pixels in a central area of the video frames.

(A3) In method (A1) or (A2), classifying further comprises: inputting the preprocessed video into a neural network model as a series of target frames, each with a plurality of preceding frames; assigning each target frame to a class of an inferred behavior; and outputting the class for each target frame.

(A4) In method (A3), wherein the neural network model is a 3D convoluted neural network (CNN) model.

(A5) In method (A4), classifying the video frame-by-frame using a target frame and a plurality of frames preceding the target frame.

(A6) In any of methods (A1-A3), the neural network model is a SlowFast model.

(A7) In any of methods (A1-A3), wherein aggregating frames further comprises: determining how many frames in a section of video are assigned to the inferred behavior; and if the number of frames is greater than a threshold, assigning the inferred behavior to the section of video.

(A8) In method (A7), wherein the threshold is 10% of a number of frames in the section of video.

(A9) In method (A7), wherein the section of video is one minute of video.

(A10) A method of detecting health-related behaviors, comprising training a model using the method of any of methods (A1-A9), capturing video using a camera focused on a user's mouth; processing the video using the model; and outputting health-related behaviors detected in the captured video by the model.

(B1) A wearable device for inferring eating behaviors in real-life situations, comprising a housing adapted to be worn on a user's head: a camera attached to the housing, the camera positioned to capture a video of a mouth of the user; a processor for processing the video; a memory for storing the video and instructions for processing the video: wherein the processor executes instructions stored in the memory to: preprocess a video captured by a camera focused on a user's mouth; classify the video frame-by-frame using a target frame and a plurality of frames preceding the target frame: aggregate video frames in sections based on their classifications; and output an inferred eating behavior of each segment of the captured video.

(B2) In the wearable device of (B1), a portable power supply for providing power to the camera, processor, and memory.

(B3) In the wearable device of (B1) or (B2), wherein the housing further comprises a hat with a bill or brim extending outward from a forehead of the user.

(B4) In the wearable device of (B3), wherein the camera is mounted on the bill or brim so that it captures a view of the mouth of the user.

(B5) In the wearable device of (B1-B4), a port or antenna for downloading the results.

It should thus be noted that the matter contained in the above description or shown in the accompanying drawings should be interpreted as illustrative and not in a limiting sense. Herein, and unless otherwise indicated: (a) the adjective “exemplary” means serving as an example, instance, or illustration, and (b) the phrase “in embodiments” is equivalent to the phrase “in certain embodiments,” and does not refer to all embodiments. The following claims are intended to cover all generic and specific features described herein, as well as all statements of the scope of the present method and system, which, as a matter of language, might be said to fall therebetween.

1. A method of training a model to detect health-related behaviors, comprising
preprocessing a video captured by a camera focused on a user's mouth by extracting raw video frames and optical flow features;
classifying the video frame-by-frame;
aggregating video frames in sections based on their classifications; and
training the model using the classified and aggregated video frames.

2. The method of claim 1, wherein preprocessing further comprises, before extracting raw video frames and optical flow features;
down-sampling the video to reduce a number of frames per second; and
resizing the video to a square of pixels in a central area of the video frames.

3. The method of claim 1, wherein classifying further comprises;
inputting the preprocessed video into a neural network model as a series of target frames, each with a plurality of preceding frames;
assigning each target frame to a class of an inferred behavior; and
outputting the class for each target frame.

4. The method of claim 3, wherein the neural network model is a 3D convoluted neural network (CNN) model.

5. The method of claim 4, further comprising classifying the video frame-by-frame using a target frame and a plurality of frames preceding the target frame.

6. The method of claim 3, wherein the neural network model is a SlowFast model.

7. The method of claim 3, wherein aggregating frames further comprises;
determining how many frames in a section of video are assigned to the inferred behavior; and
if the number of frames is greater than a threshold, assigning the inferred behavior to the section of video.

8. The method of claim 7, wherein the threshold is 10% of a number of frames in the section of video.

9. The method of claim 7, wherein the section of video is one minute of video.

10. A method of detecting health-related behaviors, comprising
training a model using the method of claim 1;
capturing video using a camera focused on a user's mouth;
processing the video using the model; and
outputting one or more health-related behaviors detected in the captured video by the model.

11. A wearable device for inferring eating behaviors in real-life situations, comprising:
a housing adapted to be worn on a user's head;
a camera attached to the housing, the camera positioned to capture a video of a mouth of the user;
a processor for processing the video;
a memory for storing the video and instructions for processing the video;
wherein the processor executes instructions stored in the memory to;
preprocess a video captured by a camera focused on a user's mouth;
classify the video frame-by-frame using a target frame and a plurality of frames preceding the target frame;
aggregate video frames in sections based on their classifications; and
output an inferred eating behavior of each segment of the captured video.

12. The wearable device of claim 11, further comprising a portable power supply for providing power to the camera, processor, and memory.

13. The wearable device of claim 11, wherein the housing further comprises a hat with a bill or brim extending outward from a forehead of the user.

14. The wearable device of claim 13, wherein the camera is mounted on the bill or brim so that it captures a view of the mouth of the user.

15. The wearable device of claim 11, further comprising a port or antenna for downloading the results.

16. The wearable device of claim 12, wherein the processor further executes instructions stored in the memory to minimize power consumption by the wearable device.

17. The wearable device of claim 11, wherein the processor and memory are attached to the housing at a location different from that of the camera.

18. The wearable device of claim 17, wherein the processor and memory are attached to the wearable device at the back of a user's head.

19. The wearable device of claim 11, wherein computational resources of the processor are capable of executing the instructions in the wearable device.
